(19) United States
(12) Patent Application Publication
(10) Pub. No.: US 2024/0164809 A1
(43) Pub. Date: May 23, 2024

(54) BREAST IMPLANT REMOVAL DEVICE AND RELATED METHODS

(71) Applicant: KIRN MEDICAL DESIGN, LLC, Lexington, KY (US)

(72) Inventors: David S. KIRN, Lexington, KY (US); Kevin J. MAUDSLEY, Lexington, KY (US)

(21) Appl. No.: 17/991,283

(22) Filed: Nov. 21, 2022

(51) Int. Cl. A61B 17/34; A61F 2/12

(52) U.S. Cl. CPC A61B 17/3468; A61F 2/12; A61B 2017/00792

(57) ABSTRACT

A device for removing a breast implant, fill material, and/or a capsule from a patient is provided. The device includes a housing having proximal and distal portions. Together, the distal and proximal portions define an interior chamber for receiving the breast implant, fill material, and/or capsule. A rigid ring fixed to the proximal portion defines a funnel for deforming and guiding the breast implant, fill material, and/or capsule toward an inlet of the interior chamber. The funnel has a tapering inner diameter toward a distal end of the funnel where the inner diameter is narrowest. The device further includes a suction port and each of the proximal and distal portions of the housing provide at least 25% of the total volume of the interior chamber. In other embodiments, flared ends of the housing replace the ring and perform the same or similar functions.

### FIELD OF THE INVENTION

This document relates generally to breast prostheses or implants and, more specifically, with devices used in removing breast implants.

### BACKGROUND OF THE INVENTION

This document relates generally to breast prostheses or implants and, more specifically, with devices used in removing breast implants Breast implants, also known as breast prostheses, have been successfully utilized since the 1960's. The implants consist of an outer elastomeric shell and a fill material. In most implants, the fill material is a silicone polymer. From time to time, it may become necessary to remove a breast implant. The most often encountered reason for doing so is a malfunction of the implant, for example, a leak of the silicone fill material outside of the outer shell.

As time progresses, the outer elastomer shell of some breast implants may deteriorate allowing the fill material to leak out. This process is commonly known as gel bleed. In other circumstances, a frank disruption of the outer elastomeric shell occurs leading to direct leakage of the fill material. Other circumstances can also be indications for implant removal including, but not limited to, capsular contracture, implant malposition, a patient's desire for size change, and/or a patient's desire for removal.

Regardless of the indication for removal, actually removing a breast implant from a scar pocket, commonly known as a capsule, which naturally forms and surrounds the breast implant can present a technical and often time-consuming challenge in the operation room. More specifically, the breast implant must be brought out through a small opening in the scar pocket and skin and, preferably, removed intact. Grasping the implant digitally or with an instrument such as a clamp can lead to, or enlarge, an existing tear in the outer elastomeric shell of the implant. This is particularly true in cases of ruptured or leaking implants where the outer shell can be extremely fragile and/or partially or full disintegrated. In such cases, removal of a ruptured or leaking breast implant and leaked material can require significant amounts of operating room time to complete.

The difficulty of removal is exacerbated by the fill material, which is commonly a silicone polymer and has a tendency to adhere to everything including, for example, breast tissue, skin, surgeon's gloves, and/or surgical instruments. As such, the silicone fill material is difficult to work with and contain and requires a significant effort to clean from breast tissue and surgical instruments. Further complicating matters, fill material or gel left within the breast pocket can lead to granuloma formation which can create palpable and visible irregularities.

In one attempt to overcome the above-noted issues relating to removal of a leaking or ruptured breast implant, a liter saline bottle is utilized as a housing for receiving the removed implant. Specifically, an incision smaller than a mouth of the bottle is made in a breast capsule. The mouth of the bottle is extended through an incision in the skin and positioned over the incision in the capsule and, subsequently, a vacuum is applied at an opposite end of the bottle. The vacuum pressure is intended to draw the breast implant, including any free fill material from a ruptured or leaking breast implant, through the incisions and into the bottle. Subsequent to removal of the implant, a pocket within the capsule is packed with a sponge to assist in removal of the capsule, i.e., to assist in the performance of a capsulectomy.

While this relatively crude device is often sufficient to remove the breast implant and any free fill material, certain issues remain. First, placement of the mouth of the bottle over the smaller incision makes forming and maintaining a vacuum seal difficult for the user. Even when a successful seal is initially formed, the initial seal is easily broken during the removal process. This is primarily due to the unrestrained nature and resulting unpredictable movement of the mouth, or end, of the bottle.

In other words, the mouth of the bottle is not secured within either incision allowing the mouth to move freely during the procedure. Such movement can be caused by the user, forces associated with the vacuum created within the bottle, and/or retentive forces caused by the implant encountering the incision in the capsule and possibly the incision in the skin. Frequently, the capsule is inelastic and at times calcified making the capsule hard and brittle such that the capsule incision may not open sufficiently to allow for simple egress of the breast implant and fill material.

The movement of the bottle is further exacerbated by the collapsing nature of the bottle sidewalls which make grasping the bottle during the procedure difficult at best. The result is often a need for multiple attempts to remove the breast implant, including any leaked fill material, and the potential spreading of leaked fill material within the surgical area. Depending on the size of the incision in the patient's skin, there is further risk of tearing the incision due to the uncontrolled evacuation of the implant unless an oversized incision is utilized in which case maintaining the initial seal is made more difficult. This combination of issues can result in inconsistent results and possibly unnecessary harm to the patient.

In yet another attempt to overcome the issues relating to removal of a leaking or ruptured breast implant, an implant removal device disclosed in U.S. Published Patent App. No. 2020/0281626 to Bateman et al. is provided. The Bateman device includes a nozzle coupled to a hollow container. The nozzle, which has a neck with a constant diameter extending from the container, includes a plurality of gripping and recessed portions that are brought into contact with the implant. The plurality of gripping and recessed portions function to aid in holding or gripping the implant during the removal process. Similar to the neck, the hollow container itself maintains a constant diameter throughout which, like the neck, retards progress of the implant during removal due to frictional forces created through contact with the container, and the neck. While functioning to remove an implant, the Bateman device suffers from certain drawbacks associated with the retardation forces created by the constant diameter neck and, subsequently, the constant diameter container as well.

Accordingly, a need exists for a device and related method of efficiently and consistently removing an intact breast implant, a ruptured or leaking breast implant and leaked fill material, an intact breast implant and capsule, and/or a ruptured or leaking breast implant, leaked fill material, and capsule without further harm to the patient. Minimizing the retardation forces encountered by the implant during the removal process by utilizing varying diameters further serves to meet these objectives. The ability to remove the breast implant from the device subsequent to removal from the patient would provide the added benefit of allowing for inspection of the removed implant and identification of any markings thereon or to determine its volume.

### SUMMARY OF THE INVENTION

In accordance with the purposes and benefits described herein, a device for removing a breast implant, fill material, and/or a capsule from a patient is provided. The device includes a housing having proximal and distal portions. Together, the distal and proximal portions define an interior chamber for receiving the breast implant, fill material, and/or capsule. A rigid ring fixed to the proximal portion defines a funnel for deforming and guiding the breast implant, fill material, and/or capsule toward an inlet of the interior chamber. The funnel has a tapering inner diameter toward a distal end of the funnel where the inner diameter is narrowest. The device further includes a suction port.

In another possible embodiment, an inner diameter of the proximal portion of the housing increases from the inlet of the interior chamber to a distal end of the proximal portion of the housing.

In yet another possible embodiment, an inner diameter of the distal portion of the housing decreases from a proximal end of the distal portion of the housing to a distal end of the distal portion of the housing.

In one other possible embodiment, the inner diameters of the distal end of the proximal portion of the housing and the proximal end of the distal portion of the housing are the same.

In still another possible embodiment, the proximal and distal portions of the housing are separable and are separated by a connecting portion having an inner diameter substantially the same from a proximal end to a distal end of the central portion.

In yet another possible embodiment, a narrowest inner diameter of the proximal portion of the housing is substantially the same as the narrowest inner diameter of the funnel and increases from a proximal end of the proximal portion toward a distal end of the proximal portion.

In one other possible embodiment, each of the proximal and distal portions of the housing provide at least 25% of the total volume of the interior chamber.

In an additional embodiment, a device for removing a breast implant, fill material, and/or a capsule from a patient includes a housing having proximal and distal portions which together define an interior chamber for receiving the breast implant, fill material, and/or capsule, a ring attached to the proximal portion that defines a funnel for guiding the breast implant, fill material, and/or capsule toward an inlet of the interior chamber, and a suction port. In this embodiment, an inner diameter of the distal portion of the housing decreases from a proximal end toward a distal end of the distal portion of the housing.

In another possible embodiment, inner diameters of a distal end of the proximal portion of the housing and the proximal end of the distal portion of the housing are the same.

In still another possible embodiment, the proximal and distal portions of the housing are separable and are separated by a connecting portion having a substantially uniform inner diameter.

In still another possible embodiment, a narrowest inner diameter of the proximal portion of the housing is substantially the same as a narrowest inner diameter of the funnel and increases from a proximal end of the proximal portion to a distal end of the proximal portion.

In one other possible embodiment, each of the proximal and distal portions provide at least 25% of the total volume of the interior chamber.

In another additional embodiment, a device for removing a breast implant, fill material, and/or a capsule from a patient is provided. The device includes a housing having proximal and distal portions separated by a central portion which together define an interior chamber having a total volume for receiving the breast implant, fill material, and/or capsule. In this embodiment, each of the proximal and distal portions of the housing providing at least 25% of the total volume of the interior chamber. A ring is attached to the proximal portion and defines a funnel having a narrowing inner diameter for guiding the breast implant, fill material, and/or capsule toward an inlet of the housing. The device further includes a suction port.

In another possible embodiment, an inner diameter of the proximal portion of the housing increases from the inlet of the housing to the central portion of the housing.

In still another possible embodiment, an inner diameter of the distal portion of the housing decreases from the central portion of the housing toward a distal end of the distal portion of the housing.

In yet another possible embodiment, the inner diameters of the distal end of the proximal portion of the housing and the proximal end of the distal portion of the housing are the same.

In one other possible embodiment, the proximal and distal portions of the housing are separable and the inner diameters of the distal end of the proximal portion of the housing and the proximal end of the distal portion of the housing are substantially the same as an inner diameter of the central portion of the housing.

In still yet another possible embodiment, a narrowest inner diameter of the proximal portion of the housing is substantially the same as a narrowest inner diameter of the funnel and increases from a proximal end of the proximal portion of the housing to a distal end of the proximal portion of the housing.

In one additional possible embodiment, a plane defines the central portion of the housing.

In still another possible embodiment, an inner diameter of the distal portion of the housing decreases from the central portion of the housing toward a distal end of the distal portion of the housing.

In the following description, there are shown and described several preferred embodiments of a device for removing a breast implant, fill material, and/or a capsule and related methods of removing the breast implant and fill material. As it should be realized, the devices and methods are capable of other, different embodiments and their several details are capable of modification in various, obvious aspects all without departing from the methods and devices as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate several aspects of the invention and together with the description serve to explain certain principles thereof. In the drawing figures:

FIG. 1 is a perspective view of a device for removing a breast implant, fill material, and/or a capsule from a patient;

FIG. 2 is a perspective view of an end of the device for removing a breast implant; fill material, and/or capsule showing a ring defining a funnel fixed to an end of a proximal portion of a housing;

FIG. 3 is a perspective view of a side of the device for removing a breast implant, fill material, and/or capsule shown in a partially disassembled state;

FIG. 4 is a partial cross section view of the proximal portion of the housing showing the funnel defined by the ring and an inlet of the housing;

FIG. 5 is a cross sectional view of the device for removing a breast implant, fill material, and/or capsule showing the ring in contact with an intact breast implant at an initial stage of the implant removal process;

FIG. 6 is a perspective view of first and second portions of the device for removing a the breast implant, fill material, and/or capsule in a nested position;

FIG. 7 is a perspective view of an alternate embodiment of a unitary breast implant, fill material, and/or capsule removal device;

FIG. 8A is a partial cross section view of the proximal portion of the housing showing the funnel defined by significantly flared ends of the proximal portion of the housing;

FIG. 8B is a partial cross section view of the proximal portion of the housing showing the funnel defined by flared ends of the proximal portion of the housing; and

FIG. 9 is a perspective view of a side of an alternate embodiment of a device for removing a breast implant, fill material, and/or capsule shown in a partially disassembled state.

Reference will now be made in detail to the present preferred embodiments of the device and related methods for removing a breast implant, fill material, and; or a capsule from a patient, examples of which are illustrated in the accompanying drawing figures, wherein like numerals are used to represent like elements.

### DETAILED DESCRIPTION

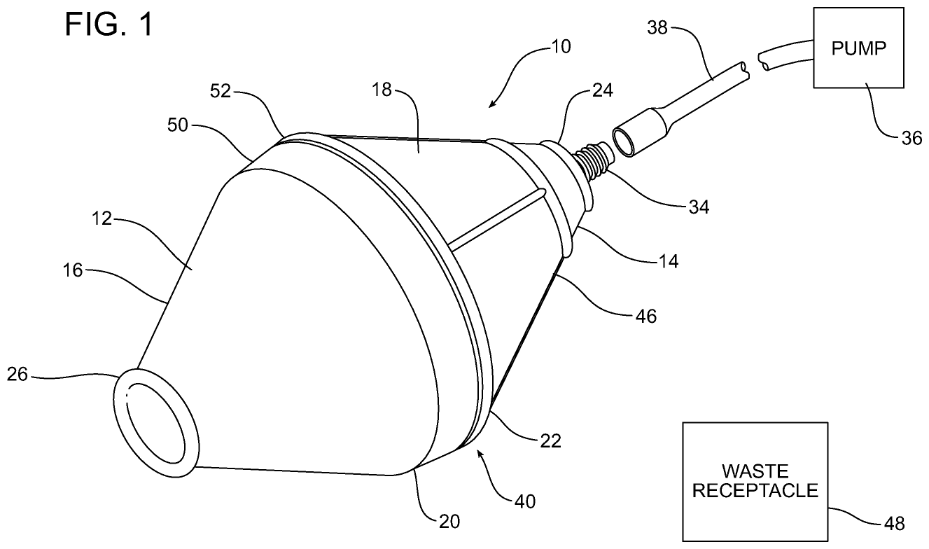

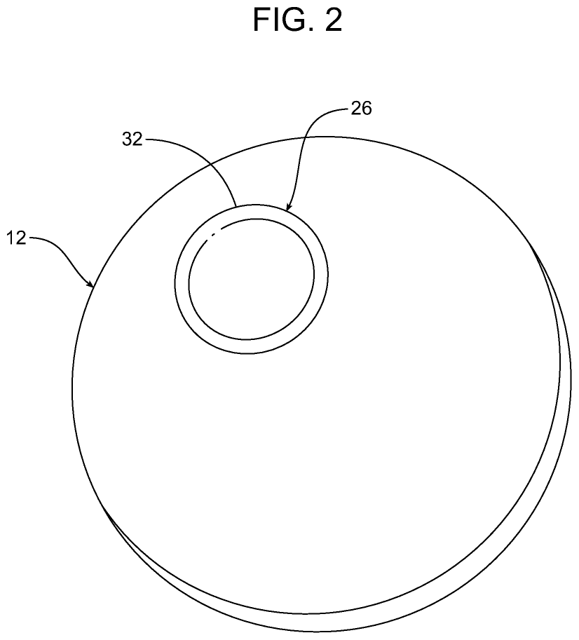

Reference is now made to FIG. 1 which illustrates a device 10 for removing a breast implant from a patient. The breast implant may be removed intact or when leaking fill material, and with or without a capsule which naturally forms at least partially around breast implants. References to breast implants within this specification refer to the breast implant including the outer elastomeric shell and the fill material, whether the shell is intact or the fill material has leaked out of the shell, and/or the capsule. In other words, a reference to contacting the breast implant includes contacting the fill material if leaked out, the outer shell, and/or the capsule unless otherwise indicated.

The device 10 consists of a first, proximal portion 12 and a second, distal portion 14 which together form a housing. In the described embodiment, the proximal and distal portions are generally halves which are separable. Each portion 12, 14 consists primarily of a solid wall 16, 18 having changing inner diameters throughout and together define an interior chamber for receiving the breast implant. Other embodiments of the device 10, however, may include a unitary housing wherein the proximal and distal portions are not separable as described below.

Each of the proximal and distal portions 12, 14 are generally frustoconical in shape, in the described embodiment, and join together to form a generally biconical housing. Of course, other shapes (e.g., round, oval, oblong, conical, at least partially cylindrical, etc.) may be utilized and the portions may take different shapes. Overall, any shape may be utilized, and the shape of the proximal and distal portions 12, 14 should be selected to accommodate the volume of the breast implant and provide sufficient mechanical strength to maintain a stable shape and avoid a collapse of the device 10 under vacuum pressure. In other words, the phrase solid wall refers to a wall of sufficient strength and/or rigidity, regardless of shape, to avoid collapse under a vacuum pressure, which is the driving force for removal or extraction of the breast implant, sufficient to remove or extract the breast implant.

The total volume of the interior chamber, adding both proximal and distal portions 12, 14 together, should be sufficient to remove large-sized breast implants. In other words, the volume of the chamber is at least 600 cc capacity or greater, in the described embodiment, to accommodate the removal of large-sized breast implants. Of course, other embodiments may utilize smaller chamber volumes and/or specifically sized chamber volumes on a patient-to-patient basis. In still other embodiments, each of the proximal and distal portions 12, 14 provide at least 25% of the total volume of the interior chamber.

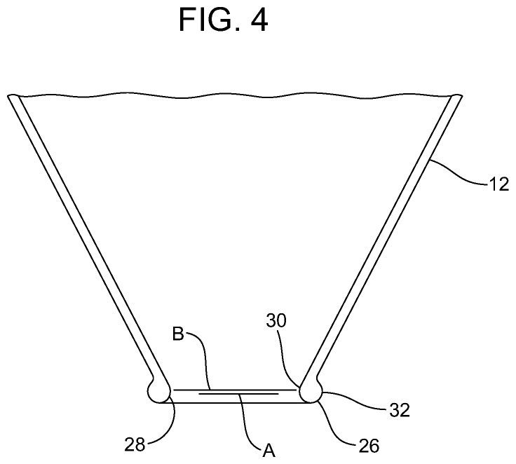

As best shown in FIG. 4, the housing includes an integrally formed ring 26 that is attached to, fixed in position relative to, and/or extending from the proximal portion 12 of the housing in the described embodiment. More specifically, the ring 26 defines a funnel (generally designated reference numeral 28) for deforming and guiding the breast implant, fill material, and/or capsule toward an inlet of the interior chamber through which the breast implant passes during removal. As shown, the funnel 28 has an inner diameter that tapers toward a distal end of the funnel where its inner diameter is narrowest. In other words, the funnel 28 terminates at its distal end which is defined by a plane perpendicular to and extending transversely across the ring 26 as represented by line A.

The inlet is similarly defined by the ring 26 essentially where a proximal end 30 of the proximal portion 12 of the housing meets the ring as defined by a second plane as represented by line B in FIG. 4. The inlet is defined by the housing/ring 26 at the proximal end 30 of the proximal portion 12 where an inner diameter of the proximal portion is narrowest. Between the distal end of funnel 28 (line A) and the inlet (line B), an inner diameter of the ring 26 continues to vary and is increasing in the described embodiment.

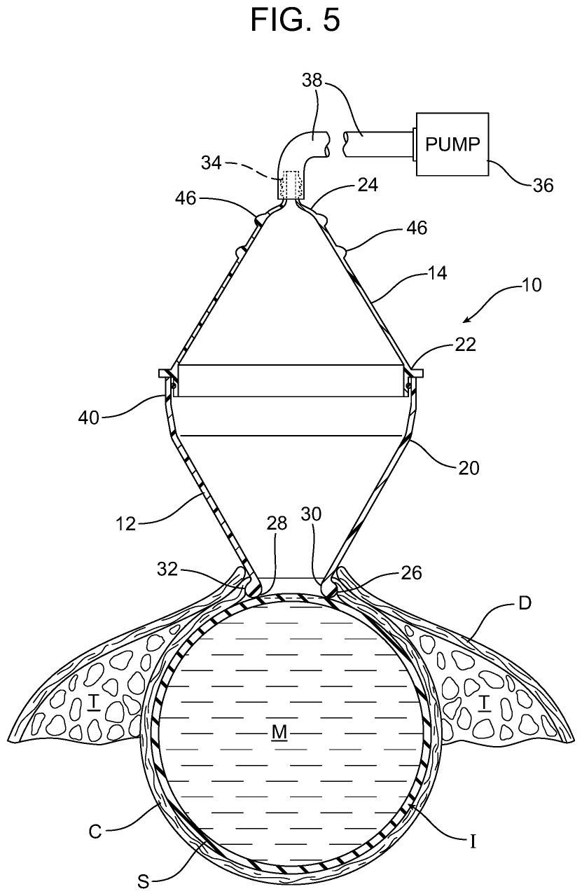

As perhaps best shown in FIG. 5, an inner diameter of the proximal portion 12 of the housing increases from the proximal end 30 to a distal end 20 of the proximal portion of the housing. This increasing inner diameter limits the amount of retardation force acting on the breast implant during the removal process. To the contrary, an inner diameter of the distal portion 14 of the housing decreases from a broadest point adjacent a connecting portion 40 (or a proximal end 22 of the distal portion 14 in some embodiments) to a distal end 24 of the distal portion of the housing. In the distal portion 14 of the housing, the decreasing inner diameter increases the retardation forces created through contact between the breast implant and the housing providing a braking function that slows the breast implant during the removal process.

As further shown, the ring 26 is positioned in contact with the breast implant (I) during the removal process such that a seal is formed between the ring and the breast implant sufficient to allow vacuum pressure to draw the breast implant from the patient, through the incision, funnel, and inlet and into the housing. In effect, the ring 26 functions as a retractor within the skin and/or capsule incisions. This positioning aids in maintaining the initial seal formed between the ring 26 and the breast implant (I) and protects the incision(s) from unwanted tearing as the implant passes through the incision(s) but within the device 10.

In other words, the rigidness of the ring 26 and funnel 28 it defines causes the breast implant (I) to alter its shape as it is guided toward and through the inlet and enters the device 10 rather than the implant or capsule being pulled through the incisions without altering shape. Depending on the patient, the capsule may calcify over time becoming hard and brittle or may be thick and inelastic limiting the elasticity of the capsule incision and the ability to accommodate egress of the breast implant. Positioning the ring 26 within the capsule incision avoids any issues relating to the elasticity of the capsule.

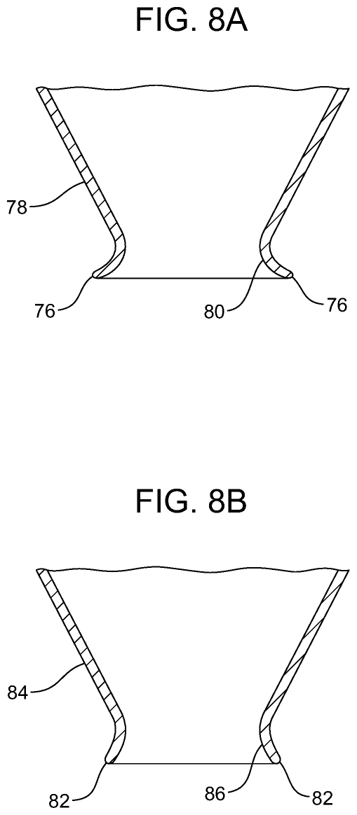

As shown, the described ring 26 includes a funnel 28 in one embodiment that is first encountered by the breast implant (I). Other embodiments, shown in FIGS. 8A and 8B for example, may not utilize a ring and the proximal portion of the housing itself may define a funnel and inlet. In all embodiments, the funnel provides a substantially smooth and mechanically advantageous ingress for the breast implant (I) being removed. Even more, the ring and funnel may be made of a lubricious material or a medical lubricant may be applied thereto to limit friction created through contact with the breast implant (I), and possibly leaked/leaking fill material (M), as the implant passes through the ring/funnel during the removal process.

The described ring 26 also includes a flared, rounded, or rolled outer lip 32 as shown in FIGS. 1, 3, and 4. The outer lip 32 helps to assist with a smooth insertion of the ring 26 into an incision in the patient's skin (D) and a capsulotomy incision as shown in FIG. 5. Further, the outer lip 32 assists with retention of the device 10 within the incision(s) and maintains edges of the incision(s) in an open position when the ring 26 is positioned in contact with the breast implant (I) during removal. In other words, the ring 26 is inserted through the incision(s) and positioned such that the ring is contacting the shell (S) of the breast implant (I) in the described embodiment. Alternatively, the ring 26 could be contacting the capsule (C) of the breast implant (I). In this position, the incision edge(s) extend around the outer lip 32 and rest around or behind the ring 26 which is generally adjacent the proximal portion 12 of the housing. While the ring 26 is described as round in one embodiment, the ring may take essentially any shape including, for example, oval or oblong. Even more, the outer lip 32 may include a flat portion adjacent the proximal portion 12 in alternate embodiments to further aid in maintaining the device 10 in position.

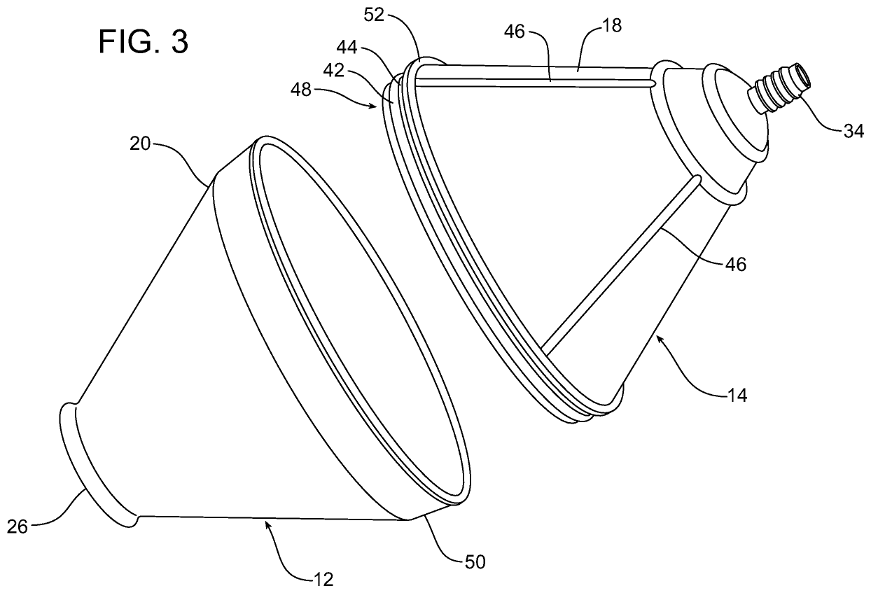

As shown in FIGS. 1 and 3, a suction port 34 extends from the distal portion 14 of the housing in the described embodiment. The suction port 34, however, may be positioned in any location on the housing. The suction port 34 is a nipple or barbed connector, in the described embodiment, designed to receive tubing connected to a suction source 36 (e.g., a vacuum pump, a syringe, and/or a hand operated pump). Various other connectors may be used to connect the device 10 to a suction source as is known in the art. In the described embodiment, the suction source 36 is a vacuum pump. Operating rooms typically provide a suction source, which may be a vacuum pump, located in a remote area or within the operating room itself. In some instances, portable vacuum pumps, syringes, or even hand operated pumps may be supplied with the device 10 for utilization in operating rooms which are void of a suitable suction source. In alternate embodiments, a tube or hose may extend from the housing with a connector attached thereto for mating with a vacuum/fluid hose or vacuum/fluid source connector. In each described instance, however, the device 10 is connected to the suction source 36 utilizing tubing 38 as shown in FIG. 1.

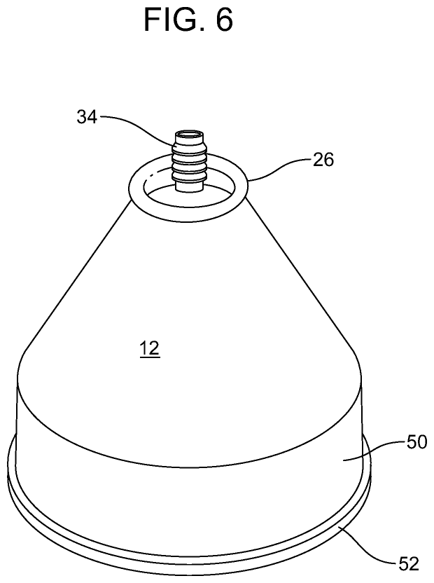

As shown and noted above, the proximal and distal portions 12, 14 are separable and generally biconical or frustroconical in shape in the described embodiment. These shapes, and others, accommodate nesting or stacking of the portions one within the other when not in use as shown in FIG. 6. In the described embodiment, the suction port 34 extends through the ring 26 when the distal and proximal portions are in the nested position. Nesting the portions in general is particularly useful for packaging the preoperative device 10. Of course, other shapes may be utilized so long as they provide sufficient mechanical rigidity to avoid collapse under vacuum pressure or the device may be a unitary structure as described above. In addition, the distal and proximal portions may be designed or a spacer inserted therebetween during shipping such that the suction port 34 extends within but not through the ring 26 or does not extend within the ring at all.

As further shown in FIGS. 1 and 3, the proximal portion 12 and the distal portion 14 are separable and are separated by a connecting portion, generally designated reference numeral 40. In the described embodiment, an inner diameter of the connecting portion 40 is substantially the same from its proximal end to its distal end. Even more, inner diameters of the distal end 20 of the proximal portion 12 of the housing and the proximal end 22 of the distal portion 14 of the housing are generally the same and are also generally the same as the inner diameter of the central portion 40 of the housing.

As shown in FIG. 3, the two portions 12, 14 of the housing are connected by a friction fit connection utilizing an O-ring 42 in the described embodiment. The connection provided by the O-ring is sufficient to maintain vacuum pressure and also prevent leaking of any silicone polymer fill material received within the device 10. More specifically, a distal portion 48 of the central portion 40 includes a gland 44 supporting the O-ring 42 in a sealing assembly. The gland 44 supports the O-ring 42 in place and provides or accommodates the necessary deformation for proper functionality. A proximal portion 50 of the central portion 40 receives the O-ring 42 and gland 44 sealing assembly of the distal portion 48 forming a connection that allows the proximal and distal portions 12, 14 to be pulled together when vacuum pressure is applied. A stop ring 52 provides a contact surface for an end of the proximal portion 50 when fully engaged. Of course, other types of mechanical connections may be utilized in lieu of the described O-ring sealing assembly in alternate embodiments such as, but not limited to, press fit connections, threaded mechanisms, butt connections with a separate connection ring, or a compression band with each sufficient to maintain the two portions 12, 14 in substantially parallel alignment to ensure a good seal.

As an added convenience, one or more grips 46 may be provided on one or both of the proximal and distal portions 12, 14 to assist the user in assembly and disassembly thereof. As best shown in FIGS. 3 and 5, the grips 46 may be elevated ridges radially extending along an outer surface of the distal portion 14. Of course, the height and length of the ridges and the number thereof may vary to provide improved gripping of the portions 12, 14 during use and/or assembly/disassembly. Of course, other types and numbers of grips may be utilized in alternate embodiments.

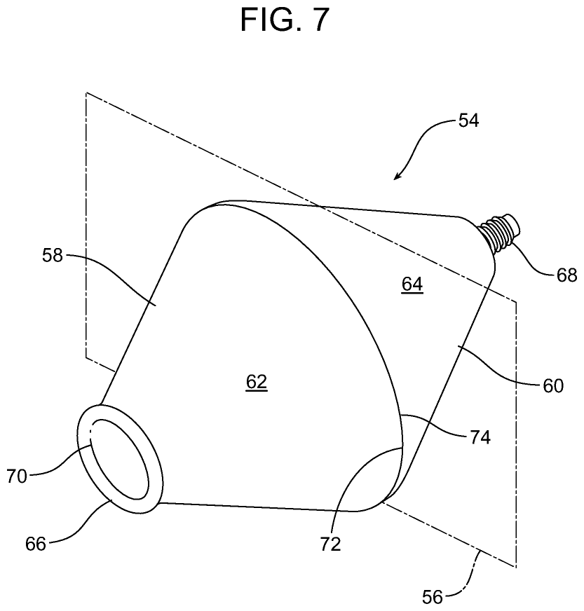

As noted above, a device 54 may be a unitary structure as shown in FIG. 7. In other words, the device 54 may be a single piece unit thereby eliminating the central or connecting portion 40 between the proximal and distal portions 12, 14 of the earlier described device 10. Described otherwise, the central portion 40 may be defined by a plane, designated reference numeral 56. The alternative device 54 similarly includes proximal and distal portions 58, 60 which define an interior chamber, and generally include solid walls, 62, 64, a ring 66, a suction port 68, and a funnel 70 in at least the described embodiment. Other than the unitary nature of the housing, the device 54 is the same as the above described embodiment and alternative embodiments thereof except a distal end 72 of the proximal portion 58 and a proximal end 74 of the distal portion 60 are essentially the same and meet at the plane 56. Even more, the unitary device 54 is intended for disposal after a single use.

In all embodiments, caps (not shown) may be provided to seal off the proximal opening and/or distal suction port prior to and/or post operation. In addition, the devices 10, 54 may be created using materials suitable for biohazardous waste disposal.

The method of removing a breast implant (I) of a patient referred to above in describing devices used therein includes the steps of exposing the breast implant, positioning a ring of the device in contact with the breast implant, and applying suction to the device sufficient to draw the breast implant through a funnel and inlet. The method is further described with reference to an embodiment of the device 10 described above.

The device 10 is supplied in a sterile condition. The user first assembles the two portions 12, 14 of the device 10 together using the O-ring 42 and gland 44 sealing assembly. Once together, the device 10 is ready for use. Surgically, a capsule (C) which naturally forms at least partially around the breast implant (I) is exposed via incisions through skin (D), breast tissue (T), and muscle, collectively the skin incision, depending on the placement of the breast implant. If a partial or total capsulectomy procedure is to be completed, the desired steps may be performed prior to or after removal of the breast implant (I). In such a scenario, the capsule or partial capsule typically accompanies the breast implant (I) into the device 10 as further described below.

As part of the described removal process, a capsulotomy incision is created in the capsule (C) thereby directly exposing the breast implant (I). The capsulotomy incision is necessarily sized large enough to admit the ring 26 in the described method. This allows for direct contact between the device 10 and the breast implant (I) which necessarily includes the outer shell (S) and/or fill material (M) if the outer shell of the breast implant has dissolved or is disrupted. More specifically, the ring 26 of the device 10 is positioned in contact with the breast implant.

In the described method, the ring 26 defines a funnel 28 and is positioned in contact with the breast implant (I) by inserting the ring through the incisions in the patient's skin (D) breast tissue (T) and capsule (C). With the ring 26 inserted through the incision in the patient's skin (D) and capsule (C) and positioned in contact with the breast implant (I), the outer lip 32 of the ring 26 assists with retention of the device 10 within the incision(s) and functions as a retractor by maintaining edges of the incision(s) in an open position. In other words, edges created by the incision in the patient's skin (D) extend around the outer lip 32 and rest behind the ring 26 during removal of the breast implant (I). Again, if an incision is made in the capsule (C), then edges of the capsule incision likewise rest behind the ring 26 which is generally adjacent the proximal portion of the housing 12.

Subsequent to inserting the ring 26 and positioning the funnel 28 in contact with the breast implant (I), a suction source 36 is connected to the suction port 34. As described above, the suction port 34 may be a nipple or barbed connector which is positioned at the distal end of the distal portion 14 of the device 10. In the described embodiment, tubing 38 is utilized to connect the suction port 34 to the suction source 36. Again, the suction source 36 may be a vacuum pump 36 provided with the device 10 or located in an operating room or in a basement of a building wherein the operating room is located. In alternative embodiments, a syringe or hand operated vacuum source may be utilized as the suction source. In other words, a suction source may be provided with the device or an existing suction source may be utilized by connecting the device thereto.

Once connected, the suction source 36 is initiated applying suction to the housing sufficient to draw the breast implant (I) through the funnel 28 and inlet. In other words, the suction source 36 is initiated drawing air out of the internal chamber of the device 10 through the tubing 38. The negative pressure created by the vacuum draws the implant (I) through the funnel 28 and inlet and into the internal chamber of the device (10). For reference, the breast implant (I) being removed will move along a proximal to distal direction during the removal process.

The natural cohesivity of the implant (I) including its outer shell (S) and fill material (M) ensure that the entire implant, including any leaked fill material or gel, is drawn into the interior chamber of the housing. In addition, if the capsule (C) has been fully mobilized from its surrounding soft tissue attachments prior to removal of the breast implant (I), the capsule typically accompanies the implant into the interior chamber. In other words, the suction applied to the housing is sufficient to draw the breast implant (I) and the capsule (C) through the funnel 28 and inlet. In such a scenario, the capsule (C) may not be breached and the ring 26 may be inserted through the incision in the patient's skin (D) and positioned in contact with the capsule.

It should be noted that the suction source 36 can be connected to the suction port 34 prior to insertion of the ring 26 and/or positioning of the ring 26 in contact with the breast implant (I) or capsule (C). Similarly, the suction source 36 may be initiated at any time throughout the process.

If the user desires to inspect the breast implant (I) now contained in the interior chamber of the device 10, then the proximal and distal portions 12, 14 may be separated opening the device and granting access to the breast implant. If the breast implant (I) is intact, it may be removed from the interior chamber for further inspection and/or disposal into a suitable biohazardous waste receptacle. The two portions 12, 14 of the device 10 may then be reassembled, and the same device may be used to remove a contralateral breast implant, if desired.

In the event the breast implant (I) is ruptured or there has been substantial gel bleed, it is recommended not to remove the breast implant from the interior chamber as doing so may expose the operative field to sticky polymer fill material (M) or gel. Rather, the breast implant (I) and device 10 may be disposed of within the biohazardous waste receptacle with or without caps applied to the ring 26 and/or distal suction port 34 as desired to seal the breast implant (I) within the interior chamber. A new removal device 10 may be used for extraction of the contralateral breast implant. In the event the one-piece alternate embodiment of the device 54 is utilized, it is preferable to use one removal device 54 per breast implant (I). In this scenario, the breast implant (I) resident in the interior chamber of the device 54 may not be taken out for inspection or otherwise.

The foregoing has been presented for purposes of illustration and description. It is not intended to be exhaustive or to limit the embodiments to the precise form disclosed. Obvious modifications and variations are possible in light of the above teachings. For instance, if a partial or total capsulectomy procedure is to be completed, the desired procedure may be performed prior to or after removal of the breast implant (I). Even more, the above-described devices may be sized to accommodate two breast implants such that removal of a first removed implant prior to removal of a contralateral implant or utilization of two devices is unnecessary. In addition, the integrally formed ring that is fixed in position and extends from the proximal portion of the housing may not be integrally formed.

In still another embodiment, the ring 26 may be integrally formed with the proximal portion 12 of the housing such that the planes, indicated by lines A and B and described above, are the same plane or are coplanar. In other words, a narrowest inner diameter of the proximal portion 12 of the housing may be substantially the same as a narrowest inner diameter of the funnel 28 and the inner diameter of the proximal portion may increase from a proximal end 30 of the proximal portion toward a distal end 20 of the proximal portion.

In still other embodiments, a ring is not utilized in favor of ends of the proximal portion itself. In such embodiments, the ends function in the same or similar manner as the ring 26 in the above-described embodiment. As shown in FIG. 8A, ends 76 extending from a proximal portion 78 are significantly flared outward and define a funnel 80 and inlet. The funnel 80 has an inner diameter that tapers toward a distal end of the funnel where its inner diameter is narrowest. The inlet is similarly defined by the ends 76 of the proximal portion 78 where an inner diameter of the proximal portion is narrowest. The funnel 80 provides a substantially smooth and mechanically advantageous ingress for the breast implant (I) being removed. The flared ends 76 also assist with retention of the device within the incision(s) and maintain edges of the incision(s) in an open position when the proximal portion 78 is positioned in contact with the breast implant (I) during removal. In other words, the proximal portion 78 is inserted through the incision(s) and positioned such that the flared ends 76 are contacting the breast implant (I) and functioning as a retractor. In this position, the incision edges extend around the flared ends 76 and rest around or behind and generally adjacent the proximal portion 78 of the housing.

Similarly, as shown in FIG. 8B, ends 82 extending from the proximal portion 84 are somewhat less significantly flared outward yet still define a funnel 86 and an inlet. The funnel 86 has an inner diameter that tapers toward a distal end of the funnel where its inner diameter is narrowest. The inlet is similarly defined by the ends 82 of the proximal portion 84 where an inner diameter of the proximal portion is narrowest. The funnel 86 provides a substantially smooth and mechanically advantageous ingress for the breast implant (I) being removed. The flared ends 82 also assist with retention of the device within the incision(s) and maintains edges of the incision(s) in an open position when the proximal portion 84 is positioned in contact with the breast implant (I) during removal. In other words, the proximal portion 84 is inserted through the incision(s) and positioned such that the flared ends 82 are contacting the breast implant (I) and functioning as a retractor. In this position, the incision edges extend around the flared ends 82 and rest around or behind and generally adjacent the proximal portion 84 of the housing. In each of the embodiments shown in FIGS. 8A and 8B, the device and its features may be the same as any of device 10 or device 54, described above, or device 90 described below.

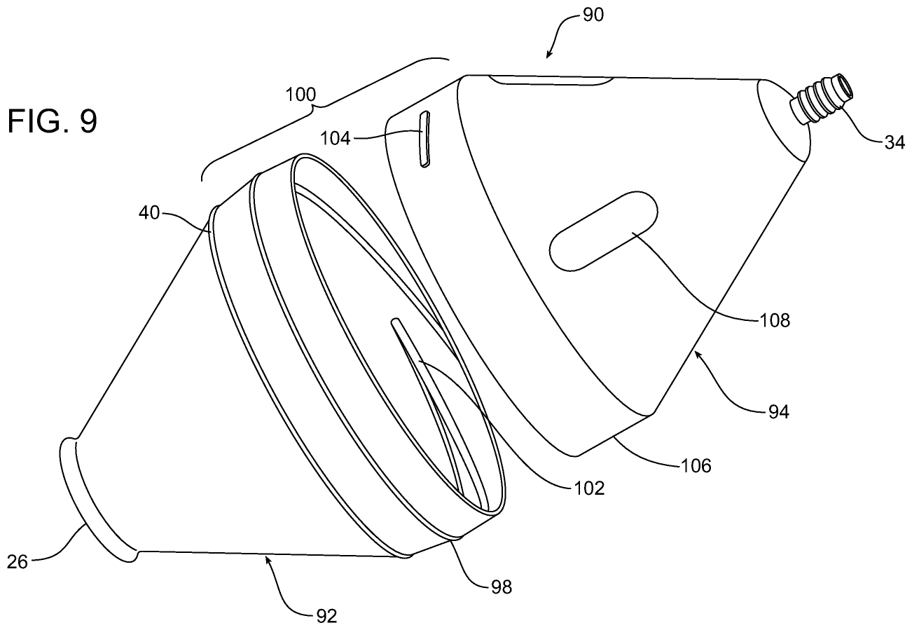

In still one other alternate embodiment shown in FIG. 9, two portions 92, 94 of the housing of a device 90 are connected by cooperating threads in the described embodiment. The connection provided by the threads is sufficient to maintain vacuum pressure and also prevent leaking of any silicone polymer fill material received within the device 90. More specifically, a proximal portion 98 of a central portion 100 has a screw thread 102 that combines with a mating screw thread 104 of a distal portion 106 of the central portion 100 forming a connection that allows the proximal and distal portions 92, 94 to be pulled together when vacuum pressure is applied.

As an added convenience, one or more grips 108 may be provided on one or both of the proximal and distal portions 92, 94 to assist the user in assembly and disassembly thereof. The grips 108 may be detents formed in the one or both portions 92, 94. While shown as generally oval in shape in the described alternate embodiment, the grips 108 may take any shape sufficient to provide improved gripping of the portions 92, 94 during use and/or assembly/disassembly. Even more, the grips may be elevated (not shown) extending above an outer surface of the housing. The elevated grips may be made of a rubber or like material and may provide a rigid and/or a tacky feel for the user.

All such modifications and variations are within the scope of the appended claims when interpreted in accordance with the breadth to which they are fairly, legally and equitably entitled.

What is claimed:

1. A device for removing a breast implant, fill material, and/or a capsule from a patient, comprising:
a housing having proximal and distal portions which together define an interior chamber for receiving the breast implant, fill material, and/or capsule;
a rigid ring fixed to the proximal portion defines a funnel for deforming and guiding the breast implant, fill material, and/or capsule toward an inlet of the interior chamber, the funnel having a tapering inner diameter toward a distal end of the funnel where the inner diameter is narrowest; and
a suction port.

2. The device for removing a breast implant, fill material, and/or a capsule from a patient of claim 1, wherein an inner diameter of the proximal portion of the housing increases from the inlet of the interior chamber to a distal end of the proximal portion of the housing.

3. The device for removing a breast implant, fill material, and/or a capsule from a patient of claim 2, wherein an inner diameter of the distal portion of the housing decreases from a proximal end of the distal portion of the housing to a distal end of the distal portion of the housing.

4. The device for removing a breast implant, fill material, and/or a capsule from a patient of claim 3, wherein the inner diameters of the distal end of the proximal portion of the housing and the proximal end of the distal portion of the housing are the same.

5. The device for removing a breast implant, fill material, and/or a capsule from a patient of claim 4, wherein the proximal and distal portions of the housing are separable and are separated by a connecting portion having an inner diameter substantially the same from a proximal end to a distal end of the central portion.

6. The device for removing a breast implant, fill material, and/or a capsule from a patient of claim 1, wherein a narrowest inner diameter of the proximal portion of the housing is substantially the same as the narrowest inner diameter of the funnel and increases from a proximal end of the proximal portion toward a distal end of the proximal portion.

7. The device for removing a breast implant, fill material, and/or a capsule from a patient of claim 1, wherein each of the proximal and distal portions of the housing provide at least 25% of the total volume of the interior chamber.

8. A device for removing a breast implant, fill material, and/or a capsule from a patient, comprising:
a housing having proximal and distal portions which together define an interior chamber for receiving the breast implant, fill material, and/or capsule;
a ring attached to the proximal portion defines a funnel for guiding the breast implant, fill material, and/or capsule toward an inlet of the interior chamber; and
a suction port, wherein an inner diameter of the distal portion of the housing decreases from a proximal end toward a distal end of the distal portion of the housing.

9. The device for removing a breast implant, fill material, and/or a capsule from a patient of claim 8, wherein inner diameters of a distal end of the proximal portion of the housing and the proximal end of the distal portion of the housing are the same.

10. The device for removing a breast implant, fill material, and/or a capsule from a patient of claim 8, wherein the proximal and distal portions of the housing are separable and are separated by a connecting portion having a substantially uniform inner diameter.

11. The device for removing a breast implant, till material, and/or a capsule from a patient of claim 8, wherein a narrowest inner diameter of the proximal portion of the housing is substantially the same as a narrowest inner diameter of the funnel and increases from a proximal end of the proximal portion to a distal end of the proximal portion.

12. The device for removing a breast implant, fill material, and/or a capsule from a patient of claim 8, wherein each of the proximal and distal portions provide at least 25% of the total volume of the interior chamber.

13. A device for removing a breast implant, fill material, and/or a capsule from a patient, comprising:
a housing having proximal and distal portions separated by a central portion which together define an interior chamber having a total volume for receiving the breast implant, fill material, and/or capsule, each of the proximal and distal portions of the housing providing at least 25% of the total volume of the interior chamber;
a ring attached to the proximal portion defines a funnel having a narrowing inner diameter for guiding the breast implant, fill material, and/or capsule toward an inlet of the housing; and
a suction port.

14. The device for removing a breast implant, fill material, and/or a capsule from a patient of claim 13, wherein an inner diameter of the proximal portion of the housing increases from the inlet of the housing to the central portion of the housing.

15. The device for removing a breast implant, fill material, and/or a capsule from a patient of claim 14, wherein an inner diameter of the distal portion of the housing decreases from a proximal end of the distal portion of the housing toward a distal end of the distal portion of the housing.

16. The device for removing a breast implant, till material, and/or a capsule from a patient of claim 15, wherein the inner diameters of the distal end of the proximal portion of the housing and the proximal end of the distal portion of the housing are the same.

17. The device for removing a breast implant, fill material, and/or a capsule from a patient of claim 16, wherein the proximal and distal portions of the housing are separable and the inner diameters of the distal end of the proximal portion of the housing and the proximal end of the distal portion of the housing are substantially the same as an inner diameter of the central portion of the housing.

18. The device for removing a breast implant, fill material, and/or a capsule from a patient of claim 13, wherein a narrowest inner diameter of the proximal portion of the housing is substantially the same as a narrowest inner diameter of the funnel and increases from a proximal end of the proximal portion of the housing to a distal end of the proximal portion of the housing.

19. The device for removing a breast implant, fill material, and/or a capsule from a patient of claim 13, wherein a plane defines the central portion of the housing.

20. The device for removing a breast implant, fill material, and/or a capsule from a patient of claim 19, wherein an inner diameter of the distal portion of the housing decreases from the central portion of the housing toward a distal end of the distal portion of the housing.
